SEQUENCE LISTING (42 sequences)

SEQ ID NO: 1 - protein, 115 aa, Artificial Sequence
  Synthetic Peptide
GSXSDXRKDXEXRFDKXVEAXKNKXDKXKAAXXKXXXXEERXKDXXKXXRKEXEQXRXAXRNXXAEXARKXSXXPTSAGXIXLXLEDXAKXXAXXXKKXEAXXADXEXXXTQXXX

SEQ ID NO: 2 - protein, 115 aa, Artificial Sequence
  Synthetic Peptide
GSRSDMRKDAERRFDKFVEAAKNKFDKFKAALRKGDIKEERRKDMKKLARKEAEQARRAVRNRLAELARKISNMPTSAGQIKLMLEDVXKFAAEAEKKIEALAADAEDKFTQGSW

SEQ ID NO: 3 - protein, 112 aa, Artificial Sequence
  Synthetic Peptide
GSMSDIRKDLEERFDKLVEALKNKVDKMKAAFRKDQFHEERMKDWFKDLRKEVEQMRRAVRNYAAEAARKISNLPTSAGDIKLALEDVXKLVAEVWKKLEAILADVEAWFTQ

SEQ ID NO: 4 - protein, 115 aa, Artificial Sequence
  Synthetic Peptide
GSLSDIRKDAERRFDKLVEAVKNKLDKMKAALRKEGQQEERMKDLMKFMRKEVEQLRKAMRNFLAEAARKISNMPTSAGDIKLILEDLXKYDAIAEKKLEAMKADVERMATQGSW

SEQ ID NO: 5 - protein, 115 aa, Artificial Sequence
  Synthetic Peptide
GSRSDMRKDAERRFDKFVEAAKNKFDKFKAALRKGDIKEERRKDMKKLARKEAEQARRAVRNRLAELARKISNMPTSAGQIKLMLEDVAKFAAEAEKKIEALAADAEDKFTQGSW

SEQ ID NO: 6 - protein, 115 aa, Artificial Sequence
  Synthetic Peptide
GSRSDMRKDAERRFDKFVEAAKNKFDKFKAALRKGDIKEERRKDMKKLARKEAEQARRAVRNRLAELARKISNMPTSAGQIKLMLEDVKKFAAEAEKKIEALAADAEDKFTQGSW

SEQ ID NO: 7 - protein, 112 aa, Artificial Sequence
  Synthetic Peptide
GSMSDIRKDLEERFDKLVEALKNKVDKMKAAFRKDQFHEERMKDWFKDLRKEVEQMRRAVRNYAAEAARKISNLPTSAGDIKLALEDVAKLVAEVWKKLEAILADVEAWFTQ

SEQ ID NO: 8 - protein, 112 aa, Artificial Sequence
  Synthetic Peptide
GSMSDIRKDLEERFDKLVEALKNKVDKMKAAFRKDQFHEERMKDWFKDLRKEVEQMRRAVRNYAAEAARKISNLPTSAGDIKLALEDVKKLVAEVWKKLEAILADVEAWFTQ

SEQ ID NO: 9 - protein, 115 aa, Artificial Sequence
  Synthetic Peptide
GSLSDIRKDAERRFDKLVEAVKNKLDKMKAALRKEGQQEERMKDLMKFMRKEVEQLRKAMRNFLAEAARKISNMPTSAGDIKLILEDLAKYDAIAEKKLEAMKADVERMATQGSW

SEQ ID NO: 10 - protein, 115 aa, Artificial Sequence
  Synthetic Peptide
GSLSDIRKDAERRFDKLVEAVKNKLDKMKAALRKEGQQEERMKDLMKFMRKEVEQLRKAMRNFLAEAARKISNMPTSAGDIKLILEDLKKYDAIAEKKLEAMKADVERMATQGSW

SEQ ID NO: 11 - protein, 115 aa, Artificial Sequence
  Synthetic Peptide
GSRSDMRKDAERRFDKFVEAAKNKFDKFKAALRKGDIKEERRKDMKKLARKEAEQARRAVRNRLAELARKISYMPTSAGQIKLMLEDVAKFAAEAEKKIEALAADAEDKFTQGSW

SEQ ID NO: 12 - protein, 115 aa, Artificial Sequence
  Synthetic Peptide
GSRSDMRKDAERRFDKFVEAAKNKFDKFKAALRKGDIKEERRKDMKKLARKEAEQARRAVRNRLAELARKISYMPTSAGQIKLMLEDVKKFAAEAEKKIEALAADAEDKFTQGSW

SEQ ID NO: 13 - protein, 112 aa, Artificial Sequence
  Synthetic Peptide
GSMSDIRKDLEERFDKLVEALKNKVDKMKAAFRKDQFHEERMKDWFKDLRKEVEQMRRAVRNYAAEAARKISYLPTSAGDIKLALEDVAKLVAEVWKKLEAILADVEAWFTQ

SEQ ID NO: 14 - protein, 112 aa, Artificial Sequence
  Synthetic Peptide
GSMSDIRKDLEERFDKLVEALKNKVDKMKAAFRKDQFHEERMKDWFKDLRKEVEQMRRAVRNYAAEAARKISYLPTSAGDIKLALEDVKKLVAEVWKKLEAILADVEAWFTQ

SEQ ID NO: 15 - protein, 115 aa, Artificial Sequence
  Synthetic Peptide
GSLSDIRKDAERRFDKLVEAVKNKLDKMKAALRKEGQQEERMKDLMKFMRKEVEQLRKAMRNFLAEAARKISYMPTSAGDIKLILEDLAKYDAIAEKKLEAMKADVERMATQGSW

SEQ ID NO: 16 - protein, 115 aa, Artificial Sequence
  Synthetic Peptide
GSLSDIRKDAERRFDKLVEAVKNKLDKMKAALRKEGQQEERMKDLMKFMRKEVEQLRKAMRNFLAEAARKISYMPTSAGDIKLILEDLKKYDAIAEKKLEAMKADVERMATQGSW

SEQ ID NO: 17 - protein, 115 aa, Artificial Sequence
  Synthetic Peptide
GSRSDMRKDAERRFDKFVEAAKNKFDKFKAALRKGDIKEERRKDMKKLARKEAEQARRAVRNRLAELARKVSNMPTSXXQXXLMXEDVXKFAAEAEKKIEALAADAEDKFTQGSW

SEQ ID NO: 18 - protein, 112 aa, Artificial Sequence
  Synthetic Peptide
GSMSDIRKDLEERFDKLVEALKNKVDKMKAAFRKDQFHEERMKDWFKDLRKEVEQMRRAVRNYAAEAARKISNLPTSXXDXXLAXEDVXKLVAEVWKKLEAILADVEAWFTQ

SEQ ID NO: 19 - protein, 115 aa, Artificial Sequence
  Synthetic Peptide
GSLSDIRKDAERRFDKLVEAVKNKLDKMKAALRKEGQQEERMKDLMKFMRKEVEQLRKAMRNFLAEAARKISNMPTSXXDXXLIXEDLXKYDAIAEKKLEAMKADVERMATQGSW

SEQ ID NO: 20 - protein, 115 aa, Artificial Sequence
  Synthetic Peptide
GSRSDMRKDAERRFDKFVEAAKNKFDKFKAALRKGDIKEERRKDMKKLARKEAEQARRAVRNRLAELARKVSNMPTSAGQIELMLEDVAKFAAEAEKKIEALAADAEDKFTQGSW

SEQ ID NO: 21 - protein, 112 aa, Artificial Sequence
  Synthetic Peptide
GSMSDIRKDLEERFDKLVEALKNKVDKMKAAFRKDQFHEERMKDWFKDLRKEVEQMRRAVRNYAAEAARKISNLPTSAGDIELALEDVAKLVAEVWKKLEAILADVEAWFTQ

SEQ ID NO: 22 - protein, 115 aa, Artificial Sequence
  Synthetic Peptide
GSLSDIRKDAERRFDKLVEAVKNKLDKMKAALRKEGQQEERMKDLMKFMRKEVEQLRKAMRNFLAEAARKISNMPTSAGDIELILEDLAKYDAIAEKKLEAMKADVERMATQGSW

SEQ ID NO: 23 - protein, 115 aa, Artificial Sequence
  Synthetic Peptide
GSRSDMRKDAERRFDKFVEAAKNKFDKFKAALCKGDIKEERRKDMKKLARKEAEQARRAVRNRLAELARKVSNMPTSAGQIKLMLEDVAKFAAEAEKKIEALAADAEDKFTQGSW

SEQ ID NO: 24 - protein, 115 aa, Artificial Sequence
  Synthetic Peptide
GSRSDMRKDAERRFDKFVEAAKNKFDKFKAALCKGDIKEERRKDMKKLARKEAEQARRAVRNRLAELARKVSNMPTSAGQIKLMLEDVKKFAAEAEKKIEALAADAEDKFTQGSW

SEQ ID NO: 25 - protein, 112 aa, Artificial Sequence
  Synthetic Peptide
GSMSDIRKDLEERFDKLVEALKNKVDKMKAAFCKDQFHEERMKDWFKDLRKEVEQMRRAVRNYAAEAARKISNLPTSAGDIKLALEDVAKLVAEVWKKLEAILADVEAWFTQ

SEQ ID NO: 26 - protein, 112 aa, Artificial Sequence
  Synthetic Peptide
GSMSDIRKDLEERFDKLVEALKNKVDKMKAAFCKDQFHEERMKDWFKDLRKEVEQMRRAVRNYAAEAARKISNLPTSAGDIKLALEDVKKLVAEVWKKLEAILADVEAWFTQ

SEQ ID NO: 27 - protein, 115 aa, Artificial Sequence
  Synthetic Peptide
GSLSDIRKDAERRFDKLVEAVKNKLDKMKAALCKEGQQEERMKDLMKFMRKEVEQLRKAMRNFLAEAARKISNMPTSAGDIKLILEDLAKYDAIAEKKLEAMKADVERMATQGSW

SEQ ID NO: 28 - protein, 115 aa, Artificial Sequence
  Synthetic Peptide
GSLSDIRKDAERRFDKLVEAVKNKLDKMKAALCKEGQQEERMKDLMKFMRKEVEQLRKAMRNFLAEAARKISNMPTSAGDIKLILEDLKKYDAIAEKKLEAMKADVERMATQGSW

SEQ ID NO: 29 - protein, 82 aa, Artificial Sequence
  Synthetic Peptide
GSMSDIRKDLEERFDKLVEALKKGQGRQKEVEQMRRAVRNYAAEAARKISNLPTSAGDIKLALEDVAKLVAEVWKKLEAILA

SEQ ID NO: 30 - protein, 82 aa, Artificial Sequence
  Synthetic Peptide
GSMSDIRKDLEERFDKLVEALKKGQGRQKEVEQMRRAVRNYAAEAARKISNLPTSAGDIKLALEDVKKLVAEVWKKLEAILA

SEQ ID NO: 31 - protein, 82 aa, Artificial Sequence
  Synthetic Peptide
GSMSDIRKDLEERFDKLVEALKKGQGRQKEVEQMRRAVRNYAAEAARKISYLPTSAGDIKLALEDVAKLVAEVWKKLEAILA

SEQ ID NO: 32 - protein, 82 aa, Artificial Sequence
  Synthetic Peptide
GSMSDIRKDLEERFDKLVEALKKGQGRQKEVEQMRRAVRNYAAEAARKISYLPTSAGDIKLALEDVKKLVAEVWKKLEAILA

SEQ ID NO: 33 - protein, 85 aa, Artificial Sequence
  Synthetic Peptide
GSLSDIRKDAERRFDKLVEAVKNLGNGEKEVEQLRKAMRNFLAEAARKISNMPTSAGDIKLILEDLAKYDAIAEKKLEAMKAGSW

SEQ ID NO: 34 - protein, 85 aa, Artificial Sequence
  Synthetic Peptide
GSLSDIRKDAERRFDKLVEAVKNLGNGEKEVEQLRKAMRNFLAEAARKISNMPTSAGDIKLILEDLKKYDAIAEKKLEAMKAGSW

SEQ ID NO: 35 - protein, 85 aa, Artificial Sequence
  Synthetic Peptide
GSLSDIRKDAERRFDKLVEAVKNLGNGEKEVEQLRKAMRNFLAEAARKISYMPTSAGDIKLILEDLAKYDAIAEKKLEAMKAGSW

SEQ ID NO: 36 - protein, 85 aa, Artificial Sequence
  Synthetic Peptide
GSLSDIRKDAERRFDKLVEAVKNLGNGEKEVEQLRKAMRNFLAEAARKISYMPTSAGDIKLILEDLKKYDAIAEKKLEAMKAGSW

SEQ ID NO: 37 - protein, 65 aa, Artificial Sequence
  Synthetic Peptide
GSCSDIRKDCERRFDKGDGGRKAMRNFLAECARKISXMPTSAGDIKLCLEDLXKYDAIAEKKGSW

SEQ ID NO: 38 - protein, 65 aa, Artificial Sequence
  Synthetic Peptide
GSLSDCRKDCERRFDKGDGGRKAMRNFLAECARCISXMPTSAGDIKLILEDLXKYDAIAEKKGSW

SEQ ID NO: 39 - protein, 62 aa, Artificial Sequence
  Synthetic Peptide
GSCSDIRKDAERRFDKGDGGRKAWRNFLAEFARKISXMPTSAGDIKLCLEDLXKYLAIAEKK

SEQ ID NO: 40 - protein, 62 aa, Artificial Sequence
  Synthetic Peptide
GSLSDCRKDAERRFDKGDGGRKAWRNFLAEFARCISXMPTSAGDIKLILEDLXKYLAIAEKK

SEQ ID NO: 41 - protein, 62 aa, Artificial Sequence
  Synthetic Peptide
GSLSDIRKDCERRFDKGDGGRKAWRNFLAECARKISXMPTSAGDIKLILEDLXKYLAIAEKK

SEQ ID NO: 42 - protein, 62 aa, Artificial Sequence
  Synthetic Peptide
GSLSDICKDAERRFDKGDGGRKAWRNFLAEFARKISXMPTSAGDIKLILEDCXKYLAIAEKK

(19) United States
(12) Patent Application Publication
(10) Pub. No.: US 2015/0044248 A1
(43) Pub. Date: Feb. 12, 2015

(54) POLYPEPTIDES AND THEIR USE IN TREATING METAPNEUMOVIRUS (MPV) INFECTION

(71) Applicant: THE SCRIPPS RESEARCH INSTITUTE, La Jolla, CA (US)

(72) Inventor: William R. Schief, Encinitas, CA (US)

(21) Appl. No.: 14/390,894

(22) PCT Filed: Apr. 4, 2013
(86) PCT No.: PCT/US13/35241

§ 371 Date: Oct. 6, 2014

Related U.S. Application Data
(60) Provisional application No. 61/621,295, filed on Apr. 6, 2012.

(51) Int. Cl. A61K 39/385; C07K 14/005; C07K 14/00; C12N 7/00

(52) U.S. Cl. CPC A61K 39/385; C12N 7/00; C07K 14/005; C07K 14/00; C12N 2760/18022; C12N 2760/18034; C07K 2319/35; C12N 2760/18023; A61K 2039/64
USPC 424/186.1; 530/350; 536/23.4; 435/320.1; 435/252.33

(57) ABSTRACT

Polypeptides and compositions thereof are provided for treating or limiting metapneumovirus (MPV) infection, as well as methods for designing such polypeptides. In further aspects, methods of using said isolated polypeptides, VLPs or pharmaceutical compositions are provided, which include methods for treating a metapneumovirus (MPV) infection, methods for limiting development of an MPV infection, methods for generating an immune response in a subject, methods for monitoring an MPV-induced disease in a subject and/or monitoring response of the subject to immunization by an MPV vaccine, methods for detecting MPV binding antibodies, methods for producing MPV antibodies, and methods of preventing an MPV infection.

### CROSS REFERENCE TO RELATED APPLICATION

This application claims the benefit of priority to U.S. Provisional Patent Application No. 61/621,295, filed on Apr. 6, 2012, which is hereby incorporated by reference in its entirety.

### SEQUENCE LISTING

The sequence listing is filed with the application in electronic format only and is incorporated by reference herein. The sequence listing text file “WO00_ASFILED_SequenceListing-Text” was created on Apr. 4, 2013 and is 47,211 bytes in size.

### INTRODUCTION

Metapneumovirus (MPV) is a respiratory viral pathogen that causes a spectrum of illness from asymptomatic infection to severe bronochiolitis. MPV is the second most common cause of lower respiratory infection in young children. MPV can also cause severe illness in the elderly and immunocompromised individuals. MPV is a member of the pneumovirus subfamily of the Paramyxoviridae. The paramyxovirus F protein is a class I viral fusion protein and a major target of the neutralizing antibody response. Major antigenic sites in human MPV have been identified, yet the understanding of F neutralizing epitopes remains incomplete. There are no known treatments for or vaccines for preventing infections caused by MPV.

### BRIEF DESCRIPTION

Compositions and methods are provided in which peptides/polypeptides may be used for preventing, attenuating, limiting and/or treating infection and disease caused by MPV. The peptides/polypeptides may also be used as a vaccine against MPV infection.

In one embodiment, an isolated polypeptide is provided comprising an amino acid sequence according to SEQ ID NO: 1.

In another embodiment, an isolated polypeptide is provided comprising an amino acid sequence according to at least one of SEQ ID NO: 17, SEQ ID NO: 18 and SEQ ID NO:19.

In a further embodiment, an isolated polypeptide is provided comprising an amino acid sequence having at least 90% amino acid sequence identity to a sequence selected from the group consisting of SEQ ID NO: 29, SEQ ID NO: 30, SEQ ID NO: 31, SEQ ID NO: 32, SEQ ID NO: 33, SEQ ID NO: 34, SEQ ID NO: 35, and SEQ ID NO: 36.

In a further embodiment, an isolated polypeptide is provided comprising an amino acid sequence according to at least one of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, and SEQ ID NO: 42.

Also provided are virus-like particles (“VLP”) including isolated polypeptides comprising an amino acid sequence according to at least one of SEQ ID NO: 1, SEQ ID NO: 17, SEQ ID NO: 18, SEQ ID NO:19, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, and SEQ ID NO: 42 or an amino acid sequence having at least 90% amino acid sequence identity to a sequence selected from the group consisting of SEQ ID NO: 29, SEQ ID NO: 30, SEQ ID NO: 31, SEQ ID NO: 32, SEQ ID NO: 33, SEQ ID NO: 34, SEQ ID NO: 35, and SEQ ID NO: 36, isolated nucleic acids encoding said isolated polypeptides, recombinant expression vectors comprising said isolate nucleic acids, recombinant host cells comprising said recombinant expression vectors, and pharmaceutical combinations comprising said isolated polypeptides, VLPs, isolated nucleic acids, recombinant expression vectors, or recombinant host cells, and a pharmaceutically acceptable carrier.

In further aspects, methods of using said isolated polypeptides, VLPs or pharmaceutical compositions are provided, which include methods for treating a metapneumovirus (MPV) infection, methods for limiting development of an MPV infection, methods for generating an immune response in a subject, methods for monitoring an MPV-induced disease in a subject and/or monitoring response of the subject to immunization by an MPV vaccine, methods for detecting MPV binding antibodies, methods for producing MPV antibodies, and methods of preventing an MPV infection.

### DETAILED DESCRIPTION

In embodiments describes herein, compositions and methods are provided in which peptides/polypeptides may be used for preventing and/or treating infection and disease caused by MPV. These peptides/polypeptides, i.e., epitope-scaffolds, may stabilize the structure of an epitope from human MPV for the purpose of isolating therapeutic anti-MPV antibodies and for inducing anti-MPV antibodies by vaccination, i.e., the peptides/polypeptides may be used as a vaccine against MPV infection.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Further, no admission is made that any reference, including any patent or patent document, cited in this specification constitutes prior art. In particular, it will be understood that, unless otherwise stated, reference to any document herein does not constitute an admission that any of these documents form part of the common general knowledge in the prior art in the United States or in any other country. Any discussion of the references states what their authors assert, and the applicant reserves the right to challenge the accuracy and pertinence of any of the documents cited herein.

Throughout this disclosure, various aspects of the methods and systems described herein may be presented in a range format. It should be understood that the description in range format is merely for convenience and brevity, and should not be construed as an inflexible limitation on the scope of the processes described herein. Accordingly, as will be understood by one skilled in the art, for any and all purposes, particularly in terms of providing a written description, all ranges disclosed herein also encompass any and all possible subranges and combinations of subranges thereof, as well as all integral and fractional numerical values within that range. As only one example, a range of 20% to 40% may be broken down into ranges of 20% to 32.5% and 32.5% to 40%, 20% to 27.5% and 27.5% to 40%, etc. Any listed range is also easily recognized as sufficiently describing and enabling the same range being broken down into at least equal halves, thirds, quarters, fifths, tenths, etc. As a non-limiting example, each range discussed herein may be readily broken down into a lower third, middle third, and upper third, etc. Further, as will also be understood by one skilled in the art, all language such as “up to,” “at least,” “greater than,” “less than,” “more than” and the like include the number recited and refer to ranges which may be subsequently broken down into subranges as discussed above. In the same manner, all ratios disclosed herein also include all subratios falling within the broader ratio. Further, the phrases “ranging/ranges between” a first indicate number and a second indicate number and “ranging/ranges from” a first indicate number “to” a second indicate number are used herein interchangeably. The foregoing are only examples of what is specifically intended.

Also, it is to be understood that the phraseology and terminology used herein is for the purpose of description and should not be regarded as limiting. The use of “including,” “including,” “having,” and variations thereof herein is meant to encompass the items listed thereafter and equivalents thereof as well as additional items. “Including” encompasses the terms “consisting of” and “consisting essentially of.” The use of “consisting essentially of” means that the composition or method may include additional ingredients and/or steps, but only if the additional ingredients and/or steps do not materially alter the basic and novel characteristics of the claimed composition or method. Unless specified or limited otherwise, the terms such as “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

All references cited are herein incorporated by reference in their entirety. Within this application, unless otherwise stated, the techniques utilized may be found in any of several well-known references such as: Molecular Cloning: A Laboratory Manual (Sambrook et al., Cold Spring Harbor Laboratory Press (1989)), Gene Expression Technology (Methods in Enzymology, Vol. 185, edited by D. Goeddel, Academic Press, San Diego, Calif. (1991)), “Guide to Protein Purification” in Methods in Enzymology (M. P. Deutshcer, ed., Academic Press, Inc. (1990)); PCR Protocols: A Guide to Methods and Applications (Innis et al. Academic Press, San Diego, Calif. (1990)), Culture of Animal Cells: A Manual of Basic Technique, 2nd Ed. (R. I. Freshney. Liss, Inc. New York, N.Y. (1987)), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V). As used herein, the singular forms “a”, “an” and “the” include plural referents unless the context clearly dictates otherwise. “And” as used herein is interchangeably used with “or” unless expressly stated otherwise.

Unless otherwise noted, technical terms are used according to conventional usage. However, as used herein, the following definitions may be useful in aiding the skilled practitioner in understanding the embodiments described herein.

As used herein, the terms “antibody” and “antibodies” refer to monoclonal antibodies, multispecific antibodies, human antibodies, humanized antibodies (fully or partially humanized), animal antibodies (in one aspect, a bird (for example, a duck or goose), in another aspect, a shark or whale, in yet another aspect, a mammal, including a non-primate (for example, a cow, pig, camel, llama, horse, goat, rabbit, sheep, hamsters, guinea pig, cat, dog, rat, mouse, etc) and a non-human primate (for example, a monkey, such as a cynomologous monkey, a chimpanzee, etc), recombinant antibodies, chimeric antibodies, single-chain Fvs (scFv), single chain antibodies, single domain antibodies, Fab fragments, F(ab′)2 fragments, disulfide-linked Fv (sdFv), and anti-idiotypic (anti-Id) antibodies (including, for example, anti-Id antibodies to antibodies of the present invention), and functionally active epitope-binding fragments of any of the above. In particular, antibodies include immunoglobulin molecules and immunologically active fragments of immunoglobulin molecules, namely, molecules that contain an antigen binding site. Immunoglobulin molecules can be of any type (for example, IgG, IgE, IgM, IgD, IgA and IgY), class (for example, IgG1, IgG2, IgG3, IgG4, IgA1 and IgA2) or subclass.

As used herein, the terms “anti-MPV antibody”, “anti-MPV antibodies”, “MPV antibody”, “MPV antibodies” and “MPV binding antibody” refer to an antibody or antigen-binding fragment which recognizes and immunospecifically binds to the MPV Fusion protein (also known as “MPV F protein”). An Example of an anti-MPV antibody is neutralizing antibody DS7 Fab, which is described in Wen et al. Nat. Struct. Mol. Biol. (2012) doi: 10.1038/nsmb.2250. [Epub ahead of print].

As used herein, “metapneumovirus” and “MPV” refer to a negative-sense, single-stranded RNA virus of the family Paramyxoviridae that causes a respiratory disease, especially in children. The term “MPV-induced disease” is intended to include any disease caused, directly or indirectly, by MPV.

As used herein, the terms “mutation” and “substitution” refer to a change in an amino acid at a particular position in a sequence.

As used herein, the terms “peptide” and “polypeptide” are used in its broadest sense to refer to a sequence of subunit amino acids. The term “MPV epitope-scaffold” refers to a polypeptide for epitope conformational stabilization and presentation to an immune system, such as a human immune system.

As used herein, the term “resurfacing” refers to a method of modifying an antibody to increase its similarity to antibody variants produced naturally in humans by replacing the surface residues of the framework region with those from a human variable region. Resurfacing may be performed as described in Correia et al., J. Mol. Biol. 405:284-297 (2011) or any related application of the concept of resurfacing.

As used herein, a “therapeutically effective amount” refers to an amount of the polypeptide that is effective for treating, attenuating and/or limiting MPV infection.

As used herein, a “virus-like particle” (also known as “VLP”) refers to a structure that in at least one attribute resembles a virus but which has not been demonstrated to be infectious.

It was recognized that a crystal structure of the anti-MPV antibody DS7 bound to a multi-domain fragment of the MPV F protein (Wen et al. Nat. Struct. Mol. Biol. (2012) doi: 10.1038/nsmb.2250. [Epub ahead of print]; PDB ID: 4DAG) demonstrates that the conformation of certain structural regions of MPV are very similar to the conformation of analogous regions in respiratory syncytial virus (RSV). Specifically, the helix-turn-helix motif of MPV residues 223 to 248 (as seen in chain A of 4DAG) aligns well to a helix-turn-helix motif of RSV residues 253 to 278 (as seen in chain B in PDB ID: 3RRR). The backbone Root Mean Square Deviation (RMSD) for that alignment is 0.66 Å. This structural concordance indicates that epitope-scaffolds that present the relevant epitope from RSV may be adapted to present the analogous epitope on MPV.

It is contemplated that polypeptides may be suitably designed to elicit neutralizing antibodies. A vaccine that elicits MPV-neutralizing antibodies is desired to protect against MPV infection. The polypeptides described herein are expected to be monomeric, highly thermostable, and to have extremely high binding affinities for MPV, which will indicate that the polypeptides stabilize the desired epitope conformation, as may be confirmed by crystal structure analysis. The polypeptides falling within the scope of this genus may elicit neutralizing antibodies against MPV and may be analyzed using methods known in the art, such as those described in Mok et al., J. Virol. 82:11410-11418 (2008).

In an aspect, isolated polypeptides provided herein, include or consist of an amino acid sequence according to the following where parentheses represent variable positions in the polypeptide, with the recited amino acid residues as alternatives in these positions:

(SEQ ID NO: 1)
GS(M/L/R)SD(I/M)RKD(L/A)E(E/R)RFDK(L/F)VEA(A/V/L)
KNK(L/F/V)DK(F/M)KAA(L/F)(R/C)K(E/G/D)(G/D/Q)(Q/
I/F)(Q/K/H)EER(M/R)KD(W/L/M)(M/K/F)K(F/L/D)(A/M/
L)RKE(V/A)EQ(L/A/M)R(K/R)A(V/M)RN(F/R/Y)(L/A)AE
(A/L)ARK(I/V)S(N/Y)(M/L)PTSAG(D/Q)I(K/E)L(I/M/A)
LED(L/V)(A/K)K(F/Y/L)(D/A/V)A(E/I)(V/A)(E/W)KK
(L/I)EA(M/L/I)(K/A/L)AD(A/V)E(R/D/A)(M/K/W)(A/F)
TQ(-/G)(-/S)(-/W).

Polypeptide species of this genus are those that are present in those polypeptides demonstrating the best range of activities. Polypeptides according to this genus are those that will be exemplified as eliciting neutralizing antibodies against MPV.

Polypeptide sequences, i.e., MPV epitope-scaffolds, are disclosed that present the MPV side-chains in the above-mentioned helix-turn-helix motif. Amino acid positions were selected for the inclusion of MPV side-chains corresponding to the positions exposed on the turn and face of the helix-turn-helix epitope (residues A225 to E246) in PDB: 4DAG. One additional MPV side-chain corresponding to A249 was added in place of lysine because this position is exposed on the scaffold near the epitope. The likelihood that such MPV scaffolds may be employed to isolate or induce MPV-neutralizing antibodies is supported by Ulbrandt et al., J. Gen. Virol. 80:7799-7806 (2006) which described two isolated monoclonal antibodies, i.e., mAb 234 and mAb 338, that neutralized all 4 sub-types of MPV potently, and Ulbrandt et al., J. Gen. Virol. 89:3113-3118 (2008) which described the mapping of the binding region of the two antibodies by selecting resistance mutations, wherein the binding region is in a region of MPV which is present on the presently disclosed MPV-epitode scaffolds.

In an embodiment, isolated polypeptides provided herein, include or consist of an amino acid sequence according to the following, where the positions having an amino acid substitution are indicated in bold:

MPV_1isea_FFL_001:
(SEQ ID NO: 2)
GSRSDMRKDAERRFDKFVEAAKNKFDKFKAALRKGDIKEERRKDMKKLAR
KEAEQARRAVRNRLAELARKISNMPTSAGQIKLMLEDV[A/K]KFAAEAE
KKIEALAADAEDKFTQGSW;
MPV_1isea_FFL_005:
(SEQ ID NO: 3)
GSMSDIRKDLEERFDKLVEALKNKVDKMKAAFRKDQFHEERMKDWFKDLR
KEVEQMRRAVRNYAAEAARKISNLPTSAGDIKLALEDV[A/K]KLVAEVW
KKLEAILADVEAWFTQ;
and
MPV_1isea_FFL_007:
(SEQ ID NO: 4)
GSLSDIRKDAERRFDKLVEAVKNKLDKMKAALRKEGQQEERMKDLMKFMR
KEVEQLRKAMRNFLAEAARKISNMPTSAGDIKLILEDL[A/K]KYDAIAE
KKLEAMKADVERMATQGSW.

In an embodiment, the polypeptides include or consist of an amino acid sequence selected from the group consisting of:

MPV_1isea_FFL_001a:
(SEQ ID NO: 5)
GSRSDMRKDAERRFDKFVEAAKNKFDKFKAALRKGDIKEERRKDMKKLAR
KEAEQARRAVRNRLAELARKISNMPTSAGQIKLMLEDVAKFAAEAEKKIE
ALAADAEDKFTQGSW;
MPV_1isea_FFL_001b:
(SEQ ID NO: 6)
GSRSDMRKDAERRFDKFVEAAKNKFDKFKAALRKGDIKEERRKDMKKLAR
KEAEQARRAVRNRLAELARKISNMPTSAGQIKLMLEDVKKFAAEAEKKIE
ALAADAEDKFTQGSW;
MPV_1isea_FFL_005a:
(SEQ ID NO: 7)
GSMSDIRKDLEERFDKLVEALKNKVDKMKAAFRKDQFHEERMKDWFKDLR
KEVEQMRRAVRNYAAEAARKISNLPTSAGDIKLALEDVAKLVAEVWKKLE
AILADVEAWFTQ;
MPV_1isea_FFL_005b:
(SEQ ID NO: 8)
GSMSDIRKDLEERFDKLVEALKNKVDKMKAAFRKDQFHEERMKDWFKDLR
KEVEQMRRAVRNYAAEAARKISNLPTSAGDIKLALEDVKKLVAEVWKKLE
AILADVEAWFTQ;
MPV_1isea_FFL_007a:
(SEQ ID NO: 9)
GSLSDIRKDAERRFDKLVEAVKNKLDKMKAALRKEGQQEERMKDLMKFMR
KEVEQLRKAMRNFLAEAARKISNMPTSAGDIKLILEDLAKYDAIAEKKLE
AMKADVERMATQGSW;
and
MPV_1isea_FFL_007b:
(SEQ ID NO: 10)
GSLSDIRKDAERRFDKLVEAVKNKLDKMKAALRKEGQQEERMKDLMKFMR
KEVEQLRKAMRNFLAEAARKISNMPTSAGDIKLILEDLKKYDAIAEKKLE
AMKADVERMATQGSW.

In an embodiment, isolated polypeptides provided herein, include or consist of an amino acid sequence according to the following sequences where the bolded amino acid corresponds to an amino acid change from N to Y at position 233 in the MPV F protein as numbered in PDB ID: 4DAG:

MPV_1isea_FFL_001(B)a:
(SEQ ID NO: 11)
GSRSDMRKDAERRFDKFVEAAKNKFDKFKAALRKGDIKEERRKDMKKLAR
KEAEQARRAVRNRLAELARKISYMPTSAGQIKLMLEDVAKFAAEAEKKIE
ALAADAEDKFTQGSW;
MPV_1isea_FFL_001(B)b:
(SEQ ID NO: 12)
GSRSDMRKDAERRFDKFVEAAKNKFDKFKAALRKGDIKEERRKDMKKLAR
KEAEQARRAVRNRLAELARKISYMPTSAGQIKLMLEDVKKFAAEAEKKIE
ALAADAEDKFTQGSW;
MPV_1isea_FFL_005(B)a:
(SEQ ID NO: 13)
GSMSDIRKDLEERFDKLVEALKNKVDKMKAAFRKDQFHEERMKDWFKDLR
KEVEQMRRAVRNYAAEAARKISYLPTSAGDIKLALEDVAKLVAEVWKKLE
AILADVEAWFTQ;
MPV_1isea_FFL_005(B)b:
(SEQ ID NO: 14)
GSMSDIRKDLEERFDKLVEALKNKVDKMKAAFRKDQFHEERMKDWFKDLR
KEVEQMRRAVRNYAAEAARKISYLPTSAGDIKLALEDVKKLVAEVWKKLE
AILADVEAWFTQ;
MPV_1isea_FFL_007(B)a:
(SEQ ID NO: 15)
GSLSDIRKDAERRFDKLVEAVKNKLDKMKAALRKEGQQEERMKDLMKFMR
KEVEQLRKAMRNFLAEAARKISYMPTSAGDIKLILEDLAKYDAIAEKKLE
AMKADVERMATQGSW;
and
MPV_1isea_FFL_007(B)b:
(SEQ ID NO: 16)
GSLSDIRKDAERRFDKLVEAVKNKLDKMKAALRKEGQQEERMKDLMKFMR
KEVEQLRKAMRNFLAEAARKISYMPTSAGDIKLILEDLKKYDAIAEKKLE
AMKADVERMATQGSW.

In an aspect, the polypeptide is an MPV epitope-scaffold with a single cysteine residue added to facilitate conjugation to particles or to facilitate targeted biotinylation. Specific examples of such molecules include the following with the C in bold.

MPV_1isea_FFL_001_R33Ca:
(SEQ ID NO: 23)
GSRSDMRKDAERRFDKFVEAAKNKFDKFKAALCKGDIKEERRKDMKKLAR
KEAEQARRAVRNRLAELARKVSNMPTSAGQIKLMLEDVAKFAAEAEKKIE
ALAADAEDKFTQGSW;
MPV_1isea_FFL_001_R33Cb:
(SEQ ID NO: 24)
GSRSDMRKDAERRFDKFVEAAKNKFDKFKAALCKGDIKEERRKDMKKLAR
KEAEQARRAVRNRLAELARKVSNMPTSAGQIKLMLEDVKKFAAEAEKKIE
ALAADAEDKFTQGSW;
MPV_1isea_FFL_005_R33Ca:
(SEQ ID NO: 25)
GSMSDIRKDLEERFDKLVEALKNKVDKMKAAFCKDQFHEERMKDWFKDLR
KEVEQMRRAVRNYAAEAARKISNLPTSAGDIKLALEDVAKLVAEVWKKLE
AILADVEAWFTQ;
MPV_1isea_FFL_005_R33Cb:
(SEQ ID NO: 26)
GSMSDIRKDLEERFDKLVEALKNKVDKMKAAFCKDQFHEERMKDWFKDLR
KEVEQMRRAVRNYAAEAARKISNLPTSAGDIKLALEDVKKLVAEVWKKLE
AILADVEAWFTQ;
MPV_1isea_FFL_007_R33Ca:
(SEQ ID NO: 27)
GSLSDIRKDAERRFDKLVEAVKNKLDKMKAALCKEGQQEERMKDLMKFMR
KEVEQLRKAMRNFLAEAARKISNMPTSAGDIKLILEDLAKYDAIAEKKLE
AMKADVERMATQGSW
and
MPV_1isea_FFL_007_R33Cb:
(SEQ ID NO: 28)
GSLSDIRKDAERRFDKLVEAVKNKLDKMKAALCKEGQQEERMKDLMKFMR
KEVEQLRKAMRNFLAEAARKISNMPTSAGDIKLILEDLKKYDAIAEKKLE
AMKADVERMATQGSW.

In some embodiments, the MPV epitope scaffold are smaller scaffolds than the ones described above, i.e., shorter in amino acid length compared to any one of SEQ ID NOs: 1-28. Examples of such smaller scaffolds may include or consist of one of the following amino acid sequences, wherein the bolded amino acids indicate the amino acids selected for the inclusion of MPV side-chains corresponding to the positions exposed on the turn and face of the helix-turn-helix epitope, as described for SEQ ID NOs: 2-4:

MPV_005_mina:
(SEQ ID NO: 29)
GSMSDIRKDLEERFDKLVEALKKGQGRQKEVEQMRRAVRNYAAEAARKIS
NLPTSAGDIKLALEDVAKLVAEVWKKLEAILA;
MPV_005_minb:
(SEQ ID NO: 30)
GSMSDIRKDLEERFDKLVEALKKGQGRQKEVEQMRRAVRNYAAEAARKIS
NLPTSAGDIKLALEDVKKLVAEVWKKLEAILA;
MPV_005_min_Ba:
(SEQ ID NO: 31)
GSMSDIRKDLEERFDKLVEALKKGQGRQKEVEQMRRAVRNYAAEAARKIS
YLPTSAGDIKLALEDVAKLVAEVWKKLEAILA;
MPV_005_min_Bb:
(SEQ ID NO: 32)
GSMSDIRKDLEERFDKLVEALKKGQGRQKEVEQMRRAVRNYAAEAARKIS
YLPTSAGDIKLALEDVKKLVAEVWKKLEAILA;
MPV_007_min_Aa:
(SEQ ID NO: 33)
GSLSDIRKDAERRFDKLVEAVKNLGNGEKEVEQLRKAMRNFLAEAARKIS
NMPTSAGDIKLILEDLAKYDAIAEKKLEAMKAGSW;
MPV_007_minAb:
(SEQ ID NO: 34)
GSLSDIRKDAERRFDKLVEAVKNLGNGEKEVEQLRKAMRNFLAEAARKIS
NMPTSAGDIKLILEDLKKYDAIAEKKLEAMKAGSW;
MPV_007_min_Ba:
(SEQ ID NO: 35)
GSLSDIRKDAERRFDKLVEAVKNLGNGEKEVEQLRKAMRNFLAEAARKIS
YMPTSAGDIKLILEDLAKYDAIAEKKLEAMKAGSW;
and
MPV_007_min_Bb:
(SEQ ID NO: 36)
GSLSDIRKDAERRFDKLVEAVKNLGNGEKEVEQLRKAMRNFLAEAARKIS
YMPTSAGDIKLILEDLKKYDAIAEKKLEAMKAGSW.

In some embodiments, the MPV epitope scaffold may include or consists of one of the following amino acid sequences, which includes the amino acid change from N to Y at position 233 in the MPV F protein as numbered in PDB ID: 4DAG, as described for SEQ ID NOs: 11-16:

MPV_T_007_23L_D5:
(SEQ ID NO: 37)
GSCSDIRKDCERRFDKGDGGRKAMRNFLAECARKIS[N/Y]MPTSAGDIK
LCLEDL[A/K]KYDAIAEKKGSW;
MPV_T_007_23L_D6:
(SEQ ID NO: 38)
GSLSDCRKDCERRFDKGDGGRKAMRNFLAECARCIS[N/Y]MPTSAGDIK
LILEDL[A/K]KYDAIAEKKGSW;
MPV_T2_007_2_D1:
(SEQ ID NO: 39)
GSCSDIRKDAERRFDKGDGGRKAWRNFLAEFARKIS[N/Y]MPTSAGDIK
LCLEDL[A/K]KYLAIAEKK;
MPV_T2_007_2_D2:
(SEQ ID NO: 40)
GSLSDCRKDAERRFDKGDGGRKAWRNFLAEFARCIS[N/Y]MPTSAGDIK
LILEDL[A/K]KYLAIAEKK;
MPV_T2_007_2_D3:
(SEQ ID NO: 41)
GSLSDIRKDCERRFDKGDGGRKAWRNFLAECARKIS[N/Y]MPTSAGDIK
LILEDL[A/K]KYLAIAEKK;
and
MPV_T2_007_2_D4:
(SEQ ID NO: 42)
GSLSDICKDAERRFDKGDGGRKAWRNFLAEFARKIS[N/Y]MPTSAGDIK
LILEDC[A/K]KYLAIAEKK.

In an aspect, the polypeptide is an MPV epitope-scaffold variant of any one of SEQ ID NOs: 1-16 and 23-42 that have mutations within the MPV epitope that are possible resistance mutants from anti-MPV antibodies. MPV epitope scaffolds harboring escape mutants may be employed to isolate or induce by vaccination novel antibodies that can neutralize MPV escape virus. The conceptual utility is that to protect against MPV strains that are resistant to anti-MPV antibody neutralizing antibodies, or to prevent the emergence of such resistant MPV strains, it may be desirable to include in a vaccine epitope-scaffolds bearing resistance mutations within the MPV epitope. In this way, vaccination with “resistance mutant epitope-scaffolds” may induce antibodies that neutralize resistance mutant viruses, and hence, prevent the emergence of those resistance viruses. Similarly, the “resistance mutant epitope-scaffolds” may be used as reagents to isolate antibodies that neutralize resistance mutant viruses.

Ulbrandt et al., (2008) have identified several resistance mutations within this region of MPV, such as A238E, A238T, G239E, I241R, K242N, K242T, and L2455. Amino substitutions at positions such as positions 238, 239, 241, 242, 245 and 249 may be incorporated into the MPV epitope-scaffold sequences to generate resistant or escape mutant epitope-scaffolds. Examples of such sequences include SEQ ID NOs: 17-19. An amino acid substitution at position 242 (bolded) may also be included in the resistance mutant epitope-scaffolds as a amino acid substitution at the analogous position in RSV viruses was known to confer resistance to Motavizumab and Palivizumab (K272E in RSV; Zhu et al., J. Infect. Dis. 203:674-682 (2011)).

In an embodiment, isolated escape mutant epitope-scaffold polypeptides, provided herein, include or consist of an amino acid sequence according to the following:

MPV_1isea_FFL_001_resistance_mutants:
(SEQ ID NO: 17)
GSRSDMRKDAERRFDKFVEAAKNKFDKFKAALRKGDIKEERRKDMKKLAR
KEAEQARRAVRNRLAELARKVSNMPTS[A/E/T][G/E]Q[I/R][K/N/
T/E]LM[L/S]EDV[A/K]KFAAEAEKKIEALAADAEDKFTQGSW;
MPV_1isea_FFL_005_resistance_mutants:
(SEQ ID NO: 18)
GSMSDIRKDLEERFDKLVEALKNKVDKMKAAFRKDQFHEERMKDWFKDLR
KEVEQMRRAVRNYAAEAARKISNLPTS[A/E/T][G/E]D[I/R][K/N/
T/E]LA[L/S]EDV[A/K]KLVAEVWKKLEAILADVEAWFTQ;
and
MPV_1isea_FFL_007_resistance_mutants:
(SEQ ID NO: 19)
GSLSDIRKDAERRFDKLVEAVKNKLDKMKAALRKEGQQEERMKDLMKFMR
KEVEQLRKAMRNFLAEAARKISNMPTS[A/E/T][G/E]D[I/R][K/N/
T/E]LI[L/S]EDL[A/K]KYDAIAEKKLEAMKADVERMATQGSW.

In an embodiment, isolated polypeptides provided herein, include or consist of an amino acid sequence according to the following, where the positions having an amino acid substitution K242E is indicated in bold:

MPV_1isea_FFL_001_K242E:
(SEQ ID NO: 20)
GSRSDMRKDAERRFDKFVEAAKNKFDKFKAALRKGDIKEERRKDMKKLAR
KEAEQARRAVRNRLAELARKVSNMPTSAGQIELMLEDVAKFAAEAEKKIE
ALAADAEDKFTQGSW;
MPV_1isea_FFL_005_K242E:
(SEQ ID NO: 21)
GSMSDIRKDLEERFDKLVEALKNKVDKMKAAFRKDQFHEERMKDWFKDLR
KEVEQMRRAVRNYAAEAARKISNLPTSAGDIELALEDVAKLVAEVWKKLE
AILADVEAWFTQ;
and
MPV_1isea_FFL_007_K242E:
(SEQ ID NO: 22)
GSLSDIRKDAERRFDKLVEAVKNKLDKMKAALRKEGQQEERMKDLMKFMR
KEVEQLRKAMRNFLAEAARKISNMPTSAGDIELILEDLAKYDAIAEKKLE
AMKADVERMATQGSW.

In a further embodiment, the polypeptides include or consist of an amino acid sequence selected from the group consisting of SEQ ID NOs: 1-42. Each of these polypeptides is expected to be monomeric, highly thermostable, and have extremely high binding affinities for MPV F protein, which will indicate that the polypeptides may stabilize the desired epitope conformation. It is expected that a number of these polypeptides will elicit neutralizing antibodies against MPV.

In another embodiment, the polypeptide includes or consists of a sequence selected from the group consisting of SEQ ID NO: 1-4. In one embodiment, the polypeptide includes or consists of a sequence selected from the group consisting of SEQ ID NO: 5-16 and 23-28. In another embodiment, the polypeptide includes or consists of a sequence selected from the group consisting of SEQ ID NO: 17-19. In yet another embodiment, the polypeptide includes or consists of a sequence selected from the group consisting of SEQ ID NO: 20-22. In a further embodiment, the polypeptide includes or consists of a sequence selected from the group consisting of SEQ ID NO: 29-42.

In some embodiments, the polypeptide has at least 80%, e.g., at least 85%, at least 90%, at least 91%, at least 92%, at least 93%, at least 94%, at least 95%, at least 96%, at least 97%, at least 98%, at least 99% or 100%, amino acid sequence identity to SEQ ID NOs: 5, 6, 7, 8, 9, 10, 11, 12, 13, 14, 15, 16, 20, 21, 22, 23, 24, 25, 26, 27, 28, 29, 30, 31, 32, 33, 34, 35, or 36. In some embodiments, the polypeptide has no more than 5 differences, or more suitably, no more than 4, 3, 2, or 1 difference. In a further embodiment, the polypeptide includes any resurfaced version of the listed sequences. For example, the solvent-exposed side-chains that do not directly contact anti-MPV antibodies may be redesigned to alter the antigenic surface outside the MPV epitope.

In a further embodiment, the polypeptide includes any variant of the listed sequences obtained by adding one or more disulfide bonds.

The polypeptides contemplated herein may include L-amino acids, D-amino acids (which are resistant to L-amino acid-specific proteases in vivo), or a combination of D- and L-amino acids. The polypeptides described herein may be chemically synthesized or recombinantly expressed. The polypeptides may contain any suitable linker, etc., for use in any desired application, such as a peptide tag to facilitate polypeptide purification, or a T-help epitope to enhance the desired immune response. For example, two of the exemplified polypeptides discussed below include a C-terminal “GSW” to facilitate determining protein concentration, as those polypeptides did not include any other ‘W” residues. In some embodiments, the polypeptide does not include the C-terminal “GSW.”

The polypeptides may be linked to other compounds to promote an increased half-life in vivo, such as by PEGylation, HESylation, PASylation, glycosylation, or may be produced as an Fc-fusion or in deimmunized variants. Such linkage may be covalent or non-covalent as is understood by those skilled in the art.

In a further embodiment, the polypeptides of any embodiment described herein may further include a tag, such as a detectable moiety or therapeutic agent. The tag(s) may be linked to the polypeptide through covalent bonding, including, but not limited to, disulfide bonding, hydrogen bonding, electrostatic bonding, recombinant fusion and conformational bonding. Alternatively, the tag(s) may be linked to the polypeptide by means of one or more linking compounds. Techniques for conjugating tags to polypeptides are well known to the skilled artisan. Polypeptides including a detectable tag may be used, for example, as probes to isolate B cells that are specific for the epitope present in the polypeptide. However, they may also be used for other detection and/or analytical purposes. Any suitable detection tag may be used, including but not limited to enzymes, prosthetic groups, fluorescent materials, luminescent materials, bioluminescent materials, radioactive materials, positron emitting metals, and nonradioactive paramagnetic metal ions. The tag used will depend on the specific detection/analysis techniques and/or methods used, such as flow cytometric detection, scanning laser cytometric detection, fluorescent immunoassays, enzyme-linked immunosorbent assays (ELISAs), radioimmunoassays (RIAs), bioassays (e.g., neutralization assays), Western blotting applications, etc. When the polypeptides of embodiments described herein are used for flow cytometric detections, scanning laser cytometric detections, or fluorescent immunoassays, the tag may include, for example, a fluorophore. A wide variety of fluorophores useful for fluorescently labeling the polypeptides contemplated herein are known to the skilled artisan. When the polypeptides are used for in vivo diagnostic use, the tag may include, for example, magnetic resonance imaging (MRI) contrast agents, such as gadolinium diethylenetriaminepentaacetic acid, to ultrasound contrast agents or to X-ray contrast agents, or by radioisotopic labeling.

Polypeptides described herein may also include a tag, such as a linker (including but not limited to an amino acid linker such as cysteine or lysine), for binding to a particle, such as a virus-like particle. As another example, polypeptides described herein may be usefully attached to the surface of a microtiter plate for ELISA. The polypeptides of embodiments described herein may be fused to marker sequences to facilitate purification, as described in the examples that follow. Examples include, but are not limited to, the hexa-histidine tag (His-tag; e.g., LEHHHHHH), Avi-tag, the myc tag or the flag tag. In some embodiments, the tag is appended to the C-terminus for purification. In other embodiments, the tag is appended to the N-terminus for purification.

In another embodiment, a plurality of the polypeptides may be complexed to a dendrimer. Dendrimers are three dimensional, highly ordered oligomeric and/or polymeric compounds typically formed on a core molecule or designated initiator by reiterative reaction sequences adding the oligomers and/or polymers and providing an outer surface. Suitable dendrimers include, but are not limited to, “starburst” dendrimers and various dendrimer polycations. Methods for the preparation and use of dendrimers are well known to those of skill in the art.

In another embodiment, the polypeptides may be fused (via recombinant or chemical means) via their N-terminus, C-terminus, or both N- and C-termini, to an oligomerization domain. Any suitable oligomerization domain may be used. In one non-limiting embodiment, the polypeptides are fused to GCN4 variants that form trimers (hence trimers or hexamers of the fused polypeptide may be displayed). In another non-limiting embodiment, the polypeptides are fused to a fibritin foldon domain that forms trimers. In other non-limiting embodiments, the oligomerization domain may be any protein that assembles into particles, including but not limited to particles made from a (non-viral) lumazine synthase protein and particles made from (non-viral) ferritin or ferritin-like proteins.

In a further embodiment, the polypeptides may be chemically conjugated to liposomes. In one non-limiting embodiment, the liposomes contain a fraction of PEGylated lipid in which the PEG groups are functionalized to carry a reactive group, and the polypeptide is chemically linked to the reactive group on the PEG. In another non-limiting embodiment, additional immune-stimulating compounds are included within the liposomes, either within the lipid layers or within the interior. In another non-limiting embodiment, specific cell-targeting molecules are included on the surface of the liposome, including but not limited to molecules that bind to proteins on the surface of dendritic cells.

In yet another embodiment, a plurality (i.e., 2 or more; suitably at least 5, 10, 15, 20, 25, 50, 75, 90, or more copies) of the polypeptides may be present in a virus-like particle (VLP), to further enhance presentation of the polypeptide to the immune system. Virus-like particles contemplated herein do not carry genetic information encoding for the proteins of the virus-like particles. In general, virus-like particles lack a viral genome and, therefore, are noninfectious. In addition, virus-like particles may often be produced in large quantities by heterologous expression and may be easily purified. In an embodiment, the VLP includes viral proteins that may undergo spontaneous self-assembly, including but not limited to recombinant proteins of adeno associated viruses, rotavirus, recombinant proteins of Norwalk virus, recombinant proteins of alphavirus, recombinant proteins of foot and mouth disease virus, recombinant proteins of retrovirus, recombinant proteins of hepatitis B virus, recombinant proteins of tobacco mosaic virus, recombinant proteins of flock house virus, and recombinant proteins of human papillomavirus, and Qbeta bacteriophage particles. In one embodiment, the viral proteins include hepatitis B core antigen particles. In another embodiment, the VLPs are from lipid-enveloped viruses and include lipid as well as any suitable viral protein, including but not limited to proteins from chikungunya virus or hepatitis B surface antigen proteins. Methods for producing and characterizing recombinantly produced VLPs have been described for VLPs from several viruses, as reviewed in U.S. Pat. Pub. No. 20110236408; see also U.S. Pat. No. 7,229,624. It is expected that immunization in the context of a VLP with approximately 75 copies of a polypeptide including a sequence selected from the group consisting of SEQ ID NOs: 1-13 conjugated onto hepatitis B (HepB) core antigen particles will result in an increased immune response to the polypeptide.

The VLPs contemplated herein may be used as vaccines or antigenic formulations for treating or limiting MPV infection, as discussed herein. In some embodiments, the VLPs may further include other scaffolds presenting other epitopes from MPVF or MPVG proteins. In other embodiments, the VLP may further include scaffolds presenting epitopes from additional MPV proteins, such as M, N, G, and/or SH.

In another embodiment, the polypeptides may be present on a non-natural core particle, such as a synthetic polymer, a lipid micelle or a metal. Such core particles may be used for organizing a plurality of polypeptides described herein for delivery to a subject, resulting in an enhanced immune response. By way of example, synthetic polymer or metal core particles are described in U.S. Pat. No. 5,770,380, which discloses the use of a calixarene organic scaffold to which is attached a plurality of peptide loops in the creation of an “antibody mimic”, and U.S. Pat. No. 5,334,394 describes nanocrystalline particles used as a viral decoy that are composed of a wide variety of inorganic materials, including metals or ceramics. Suitable metals in this embodiment include chromium, rubidium, iron, zinc, selenium, nickel, gold, silver, and platinum. Suitable ceramic materials in this embodiment include silicon dioxide, titanium dioxide, aluminum oxide, ruthenium oxide and tin oxide. The core particles of this embodiment may be made from organic materials including carbon (diamond). Suitable polymers include polystyrene, nylon and nitrocellulose. For this type of nanocrystalline particle, particles made from tin oxide, titanium dioxide or carbon (diamond) are particularly suitable. A lipid micelle may be prepared by any means known in the art. See U.S. Pat. No. 7,229,624 and references disclosed therein.

In an aspect, isolated nucleic acids are provided which encode a polypeptide contemplated herein. The isolated nucleic acid sequence may include RNA or DNA. As used herein, “isolated nucleic acids” are those that have been removed from their normal surrounding nucleic acid sequences in the genome or in cDNA sequences. Such isolated nucleic acid sequences may include additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides described herein.

In yet another aspect, recombinant expression vectors are provided which include isolated nucleic acid of any aspect of the embodiments described herein, operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences described herein are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences may be present between a promoter sequence and the nucleic acid sequences and the promoter sequence may still be considered “operably linked” to the coding sequence. Other such control sequences include, but are not limited to, polyadenylation signals, termination signals, and ribosome binding sites. Such expression vectors may be of any type known in the art, including but not limited plasmid and viral-based expression vectors. The control sequence used to drive expression of the disclosed nucleic acid sequences in a mammalian system may be constitutive (driven by any of a variety of promoters, including but not limited to, CMV, SV40, MPV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus may be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.). The expression vector must be replicable in the host organisms either as an episome or by integration into host chromosomal DNA. In one embodiment, the expression vector includes a plasmid. However, other expression vectors are contemplated and included in embodiments herein that serve equivalent functions, such as viral vectors.

In a further aspect, host cells are provided that have been transfected with the recombinant expression vectors disclosed herein, wherein the host cells may be either prokaryotic or eukaryotic. The cells may be transiently or stably transfected. Such transfection of expression vectors into prokaryotic and eukaryotic cells may be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook et al., Cold Spring Harbor Laboratory Press (1989); Culture of Animal Cells: A Manual of Basic Technique, 2nd Ed. (R. I. Freshney. Liss, Inc. New York, N.Y. (1987)). A method of producing a polypeptide of any embodiment described herein is also provided. The method includes (a) culturing a host according to this aspect of embodiments described herein under conditions conducive to the expression of the polypeptide, and (b) optionally, recovering the expressed polypeptide. The expressed polypeptide may be recovered from the cell free extract, but suitably they are recovered from the culture medium. Methods to recover polypeptide from cell free extracts or culture medium are well known to the man skilled in the art.

In an aspect, pharmaceutical compositions (such as a vaccine), are provided that include one or more polypeptides, VLPs, nucleic acids, recombinant expression vectors, or host cells of embodiments described herein and a pharmaceutically acceptable carrier. The pharmaceutical compositions may be used, for example, in the methods described herein below. The pharmaceutical composition may include in addition to a polypeptide contemplated herein (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g., sucrose, sorbitol or trehalose. In certain embodiments, the pharmaceutical composition includes a preservative e.g., benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptides may be the sole active agent in the pharmaceutical composition, or the composition may further include one or more other agents suitable for an intended use, including but not limited to adjuvants to stimulate the immune system generally and improve immune responses overall. Any suitable adjuvant may be used. The term “adjuvant” refers to a compound or mixture that enhances the immune response to an antigen. Exemplary adjuvants include, but are not limited to, Adju-Phos, Adjumer™, albumin-heparin microparticles, Algal Glumay, Algammulin, Alum, Antigen Formulation, AS-2 adjuvant, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT(R192G), LT-OA or LT Oral Adjuvant, LT-R192G, LTK63, LTK72, MF59, MONTANIDE ISA 51, MONTANIDE ISA 720, MPL™, MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, Ribilike adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes. Selection of an adjuvant depends on the subject to be vaccinated. Suitably, a pharmaceutically acceptable adjuvant is used.

Compositions including the polypeptides may be stored in any standard form, including, e.g., an aqueous solution or a lyophilized cake. Such compositions are typically sterile when administered to cells or subjects. Sterilization of an aqueous solution is readily accomplished by filtration through a sterile filtration membrane. If the composition is stored in lyophilized form, the composition may be filtered before or after lyophilization and reconstitution.

In another aspect, methods are provided for treating, attenuating and/or limiting an MPV infection, including administering to a subject in need thereof a therapeutically effective amount of one or more polypeptides contemplated and described herein, salts thereof, conjugates thereof, VLPs thereof, or pharmaceutical compositions thereof, to treat and/or limit the MPV infection. In another embodiment, the method includes eliciting an immune response in an individual having or at risk of an MPV infection, including administering to a subject in need thereof a therapeutically effective amount of one or more polypeptides contemplated and described herein, salts thereof, conjugates thereof, VLPs thereof, or pharmaceutical compositions thereof, to generate an immune response.

When the method includes treating an MPV infection, the one or more polypeptides, VLPs, or compositions are administered to a subject that has already been infected with the MPV, and/or who is suffering from symptoms (including but not limited to lower respiratory tract infections, upper respiratory tract infections, bronchiolitis, pneumonia, fever, listlessness, diminished appetite, recurrent wheezing, and asthma) indicating that the subject is likely to have been infected with the MPV. As used herein, “treat” or “treating” means accomplishing one or more of the following: (a) reducing MPV titer in the subject; (b) limiting any increase of MPV titer in the subject; (c) reducing the severity of MPV symptoms; (d) limiting or preventing development of MPV symptoms after infection; (e) inhibiting worsening of MPV symptoms; (f) limiting or preventing recurrence of MPV symptoms in subjects that were previously symptomatic for MPV infection. In one embodiment method, polypeptides, VLPs, or compositions are used as “therapeutic vaccines” to ameliorate the existing infection and/or provide prophylaxis against infection with additional MPV virus.

When the method includes limiting an MPV infection, the one or more polypeptides, VLPs, or compositions are administered prophylactically to a subject that is not known to be infected, but may be at risk of exposure to the MPV. As used herein, “limiting” means to limit MPV infection in subjects at risk of MPV infection. Groups at particularly high risk include children under age 18 (particularly infants 3 years or younger), adults over the age of 65, and individuals suffering from any type of immunodeficiency. In this method, the polypeptides, VLPs, or compositions are used as vaccines.

The polypeptides are typically formulated as a pharmaceutical composition, such as those disclosed above, and may be administered via any suitable route, including orally, parentally, by inhalation spray, rectally, or topically in dosage unit formulations containing conventional pharmaceutically acceptable carriers, adjuvants, and vehicles. The term “parenteral”, as used herein, includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood). Dosage regimens may be adjusted to provide the optimum desired response (e.g., a therapeutic or prophylactic response). A suitable dosage range may, for instance, be 0.1 μg/kg-100 mg/kg body weight; alternatively, it may be 0.5 μg/kg to 50 mg/kg; 1 μg/kg to 25 mg/kg, or 5 μg/kg to 10 mg/kg body weight. The polypeptides may be delivered in a single bolus, or may be administered more than once (e.g., 2, 3, 4, 5, or more times) as determined by an attending physician.

In certain embodiments, the polypeptides of embodiments described herein neutralize MPV infectivity, as demonstrated in the examples that follow. In various embodiments, the polypeptides described herein prevent MPV from infecting host cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to infection of host cells by MPV in the absence of the polypeptides. Neutralization may be measured using standard techniques in the art.

In another aspect, a pharmaceutical composition is provided, which includes (a) isolated nucleic acids, recombinant expression vectors, and/or recombinant host cells described herein; and (b) a pharmaceutically acceptable carrier. In this aspect, the nucleic acids, expression vectors, and host cells of embodiments described herein may be used as polynucleotide-based immunogenic compositions, to express an encoded polypeptide in vivo, in a subject, thereby eliciting an immune response against the encoded polypeptide. Various methods are available for administering polynucleotides into animals. The selection of a suitable method for introducing a particular polynucleotide into an animal is within the level of skill in the art. Polynucleotides of embodiments described herein may also be introduced into a subject by other methods known in the art, e.g., transfection, electroporation, microinjection, transduction, cell fusion, DEAE dextran, calcium phosphate precipitation, lipofection (lysosome fusion), or a DNA vector transporter (see, e.g., Wu et al., J. Biol. Chem. 267:963-967 (1992)).

The immune response against the polypeptides, VLPs, or compositions of embodiments described herein may be generated by one or more inoculations of a subject with an immunogenic composition of embodiments described herein. A first inoculation is termed a “primary inoculation” and subsequent immunizations are termed “booster inoculations”. Booster inoculations generally enhance the immune response, and immunization regimens including at least one booster inoculation are most suitable. Any polypeptide, VLP, or composition of embodiments described herein may be used for a primary or booster immunization. The adequacy of the vaccination parameters chosen, e.g., formulation, dose, regimen and the like, may be determined by taking aliquots of serum from the subject and assaying antibody titers during the course of the immunization program. Alternatively, the T cell populations may by monitored by conventional methods. In addition, the clinical condition of the subject may be monitored for the desired effect, e.g., limiting MPV infection, improvement in disease state (e.g., reduction in viral load), etc. If such monitoring indicates that vaccination is sub-optimal, the subject may be boosted with an additional dose of composition, and the vaccination parameters may be modified in a fashion expected to potentiate the immune response. Thus, for example, the dose of the polypeptide, VLP, or composition, and/or adjuvant, may be increased or the route of administration may be changed.

In a further aspect, methods are provided for monitoring an MPV-induced disease in a subject and/or monitoring response of the subject to immunization by an MPV vaccine, including contacting the polypeptides, the VLPs, or pharmaceutical compositions of embodiments described herein with a bodily fluid from the subject, and detecting MPV-binding antibodies in the bodily fluid of the subject. The method includes contacting a polypeptide, VLP, or composition of embodiments described herein with an amount of bodily fluid (such as serum, whole blood, etc.) from the subject; and detecting MPV-binding antibodies in the bodily fluid of the subject. The detection of the MPV binding antibodies allows the MPV disease in the subject to be monitored. In addition, the detection of MPV binding antibody also allows the response of the subject to immunization by an MPV vaccine to be monitored. In still other methods, the titer of the MPV binding antibodies is determined. Any suitable detection assay may be used, including but not limited to homogeneous and heterogeneous binding immunoassays, such as radioimmunoassays (RIA), ELISA, immunofluorescence, immunohistochemistry, FACS, BIACORE and Western blot analyses. The methods may be carried in solution, or the polypeptide(s) of embodiments described herein may be bound or attached to a carrier or substrate, e.g., microtiter plates (ex: for ELISA), membranes and beads, etc. Carriers or substrates may be made of glass, plastic (e.g., polystyrene), polysaccharides, nylon, nitrocellulose, or teflon, etc. The surface of such supports may be solid or porous and of any convenient shape. The polypeptides contemplated and described herein for use in this aspect may include a conjugate as disclosed above, to provide a tag useful for any detection technique suitable for a given assay.

In a still further aspect, methods are provided for detecting MPV binding antibodies, including (a) contacting the polypeptides, the VLPs, or the compositions described herein with a composition including a candidate MPV binding antibody under conditions suitable for binding of MPV antibodies to the polypeptide, VLP, or composition; and (b) detecting MPV antibody complexes with the polypeptide, VLP, or composition. In this aspect, the methods are performed to determine if a candidate MPV binding antibody recognizes the MPV F epitope present in the polypeptides of embodiments described herein. Any suitable composition may be used, including but not limited to bodily fluid samples (such as serum, whole blood, etc.) from a suitable subject (such as one who has been infected with MPV), naive libraries, modified libraries, and libraries produced directly from human donors exhibiting an MPV-specific immune response. The assays are performed under conditions suitable for promoting binding of antibodies against the polypeptides; such conditions may be determined by those of skill in the art based on the teachings herein. Any suitable detection assay may be used, including but not limited to homogeneous and heterogeneous binding immunoassays, such as radioimmunoassays (RIA), ELISA, immunofluorescence, immunohistochemistry, FACS, BIACORE and Western blot analyses. The methods may be carried in solution, or the polypeptide(s) of embodiments described herein may be bound or attached to a carrier or substrate, e.g., microtiter plates (ex: for ELISA), membranes and beads, etc. Carriers or substrates may be made of glass, plastic (e.g., polystyrene), polysaccharides, nylon, nitrocellulose, or teflon, etc. The surface of such supports may be solid or porous and of any convenient shape. The polypeptides of embodiments described herein for use in this aspect may include a conjugate as disclosed above, to provide a tag useful for any detection technique suitable for a given assay. In a further embodiment, the MPV F-binding antibodies are isolated using standard procedures. In one embodiment, the methods may include isolation of polypeptide-specific memory B cells by fluorescence activated cell sorting (FACS) using standard techniques in the art (see, for example, Wu et al., Science 329:856-861 (2010)).

In another aspect, methods are provided for producing anti-MPV antibodies, which include (a) administering to a subject an amount effective to generate an antibody response of the polypeptides, the VLPs, and/or the compositions of embodiments described herein; and (b) isolating antibodies produced by the subject.

The polypeptides of embodiments described herein may also be used to generate antibodies that recognize the polypeptides described and contemplated herein. The method includes administering to a subject a polypeptide, VLP, or composition of embodiments described herein. Such antibodies may be used, for example, in MPV research. A subject employed in this embodiment is one typically employed for antibody production, including but not limited to mammals, such as, rodents, rabbits, goats, sheep, etc. The antibodies generated may be either polyclonal or monoclonal antibodies. Polyclonal antibodies are raised by injecting (e.g., subcutaneous or intramuscular injection) antigenic polypeptides into a suitable animal (e.g., a mouse or a rabbit). The antibodies are then obtained from blood samples taken from the animal. The techniques used to produce polyclonal antibodies are extensively described in the literature. Polyclonal antibodies produced by the subjects may be further purified, for example, by binding to and elution from a matrix that is bound with the polypeptide against which the antibodies were raised. Those of skill in the art will know of various standard techniques for purification and/or concentration of polyclonal, as well as monoclonal, antibodies. Monoclonal antibodies may also be generated using techniques known in the art.

All of the references cited herein are incorporated by reference. Aspects of the disclosure may be modified, if necessary, to employ the systems, functions and concepts of the above references and application to provide yet further embodiments of the disclosure. These and other changes may be made to the disclosure in light of the detailed description.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize.

Specific elements of any of the foregoing embodiments may be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following Examples, which should not be construed by way of limiting the scope of the processes described herein, further explain embodiments of the principles described herein. Moreover, all experimental processes may be further optimized for efficiency and the process of scale up is expected to achieve greater enhancement of efficiency.

### Example 1

### Expression and Purification

### Non-labeled protein

DNA segments encoding MPV epitope-scaffold polypeptide constructs (e.g., SEQ ID NO: 1-42) are synthesized with optimized codon usage and RNA structure (Codon Devices, Genscript Corp.), subcloned into pET29 (EMD Biosciences) and transformed into Arctic Express™ E. coli (Invitrogen). Single colonies are grown overnight at 37° C. in 10 mL Luria Broth (LB) plus Kanamycin (100 mg/mL). The starter cultures are expanded into 1 L of LB plus Kanamycin and incubated at 37° C.; when cells reach log phase, 250 μM of IPTG is added to the cultures to induce protein expression and the cells are then incubated overnight at 12° C. Cultures are then pelleted and resuspended in start buffer (160 mM Imidazole, 4 M Sodium Chloride, 160 mM Sodium Phosphate), a tablet of protease inhibitor (Novagen) is added and the cell suspension is frozen at −20° C.

The cell suspension is thawed and 10 mL of 10× Bugbuster™ (Novagen), 50 μL of Benzonase Nucleases and 1.7 μL of rLysozyme (Novagen) are added to lyse the cells; the cell suspension is then gently tumbled in an orbital shaker for 20 minutes. Lysed cells are pelleted and the supernatant is filtered through a 0.22 μm filter (Millipore). Supernatants are tumbled with 5 mL of Ni++ Sepharose 6 Fast Flow (GE Healthcare) for 1 hour at 4° C. The resin is washed 3 times with 30 mL wash buffer (50 mM imidazole, 500 mM Sodium Chloride and 160 mM Sodium Phosphate) and eluted with 20 mL of Elution Buffer (250 mM Imidazole, 500 mM Sodium Chloride and 20 mM Sodium Phosphate). Fractions containing the construct of interest are combined and further purified by preparative size exclusion chromatography (SEC) on Superdex 75 16/60 (GE Healthcare) at room temperature in HBS. Collected fractions are analyzed on a 4-12% SDS denaturing gel (Invitrogen) and positive fractions are combined and concentrated by ultrafiltration (Vivaspin, Bioexpress). Protein concentration is determined by measuring UV absorption signal at 280 nm (Nanodrop™) and calculated from the theoretical extinction coefficient. To facilitate a rapid and accurate protein quantification the sequence GSW is added to all the designs without tryptophans in their primary sequence.

Low-Endotoxin Protein

In order to prepare low-endotoxin protein for immunization studies, bacterial pellets are alternately resuspended in detergent buffer (50 mM NaH₂PO₄, 500 mM NaCl, 10 mM imidazole, 0.5 mg/mL lysozyme, 0.01 mg/mL DNase, 0.1% Triton X114) and Ni++ resin is alternately initially washed in 10 mM imidazole, 50 mM NaH₂PO₄, 500 mM NaCl, 0.1% Triton X114.

¹⁵N-Labeled Protein

Isotopically labeled samples of the disclosed polypeptide of any one of SEQ ID NOs: 2-13 are grown in minimal MOPS medium supplemented with 1 g/L of ¹⁵N ammonium chloride. The starter cultures are expanded to 1 L of MOPS and are incubated overnight at 37° C.; 3 mL of 40% ¹⁵N glucose is added to continue growth; upon reaching an OD_{λ=600}, 250 μM of IPTG is added to the cultures to induce protein expression and the cells are then incubated overnight at 16° C.

Light Scattering

The monodispersivity and molecular weight of purified proteins are further assessed by HPLC (Agilent, 1200 series) coupled in-line to a static light scattering device (miniDAWN TREOS™, Wyatt). 100 μL of 1-2 mg/mL protein sample is used and the collected data is analyzed with the ASTRA™ software (Wyatt).

Circular Dichroism

Solution thermostabilities (Tₘ) are determined by circular dichroism (CD) on an Aviv 62A DS spectrometer. Far-UV wavelength scans (190-260 nm) of 15 to 25 μM protein are collected in a 1 mm path length cuvette. Temperature-induced protein denaturation is followed by change in ellipticity at 210 nm. Experiments are carried over a temperature range from 1-99° C., with 2° C. increments every 3 minutes, and the resulting data is converted to mean residue ellipticity and fitted to a two-state model.

NMR

NMR samples are prepared in 25 mM sodium phosphate, 150 mM NaCl, pH 7.0, and 90% H₂O/10% D₂O at a concentration of 500 μM. Heteronuclear single quantum coherence spectra for the polypeptides are recorded on a Bruker Avance™ 600 MHz NMR spectrometer equipped with an actively shielded z-gradient triple resonance cryo-probe. All spectra are recorded at 25° C. Spectra are processed using NMRPipe™ and NMRView™.

### Example 2

The MPV epitope-scaffold polypeptide constructs are expressed in E. coli and assessed for expression and solubility. The oligomerization state in solution of these recombinant proteins is assessed by SEC and static light scatter analysis. The recombinant proteins are expected to have good yields, e.g., 3 to 5 mg/L, to be monodispersed and to exhibit an apparent molecular weight consistent with a monomeric protein. The folding and the thermal stability of the designed molecules is evaluated by CD spectroscopy and expected to show typical CD spectra of properly folded helical proteins. Temperature induced denaturation will be followed by CD and is expected to show that the stability of the designs range from about 48 to more than 100° C. The orthogonal characterization of the solution behavior and structural properties of the recombinant proteins is obtained by collecting the ¹⁵N-¹H hetero-nuclear single-quantum coherence spectra and expected to show good peak dispersion typical of protein with well-defined globular folds.

### Example 3

MPV epitope-scaffolds (e.g., SEQ ID NOs: 23-28) are conjugated to the surface of HepBcAg particles to improve immune responses to the epitope. The MPV epitope-scaffolds are conjugated via hetero-bifunctional cross-linkers between an engineered cysteine in the MPV epitope-scaffold at the opposite end from the epitope, and an engineered lysine on the tip of the major immunodominant region of HepBcAg. This orients the MPV epitope-scaffolds in such a way that the epitope is exposed at the radial exterior of the conjugated particle.

Particles from HepBcAg residues 1-149, a construct that leads to higher expression in bacteria and a predominance of the larger T=4 particle with 240 HepBcAg monomers (Zlotnick et al., Biochemistry 35:7412-7421 (1996); Wynne et al., Mol. Cell 3:771-780 (1999)) are expressed in E. coli and purified via standard sucrose gradients. For chemical coupling of monomeric immunogens, pure lysine-functionalized HepBcAg (1-149) particles are expressed and purified using standard techniques, in which a lysine residue is engineered into the tip of the immunodominant spike of every subunit. HepBcAg (1-149) WT and lysine-functionalized particles are both full size (30 nm).

Conjugation of MPV epitope-scaffolds and HepBcAG are carried out under standard conditions using a 10% Sucrose and 1% CHAPS, resulting in approximately 75 MPV epitope-scaffolds being attached to each HepB particle, according to densitometry analysis of SDS-PAGE gels run on purified fractions from sucrose gradient ultracentrifugation.

A macaque immunization experiment with the polypeptide scaffold monomers and the MPV epitope-scaffold-conjugated-HepBcAg-particles is performed. Immunogens are MPV epitope-scaffold monomers and MPV epitope-scaffold-conjugated-HepBcAg-particles. Rhesus macaques (4 animals per immunogen) are immunized by the intramuscular route at 0, 1 and 2 months. Animals are injected with 1 mL total volume of antigen mixed with Adjuplex™ adjuvant, with 0.5 mL injected into each arm. The first immunization includes a total of 200 μg of scaffold; subsequent immunizations include a total of 100 μg scaffold. “Naïve” sera are taken from each animal on day 0 before the first immunization. “Imm3” sera are taken from each animal 2 weeks after the 3^{rd} immunization. Both the “Naïve” and the “Imm3” sera are evaluated for neutralization in a standard plaque reduction assay at a serum dilution of 1:20. Each sample is run in duplicate. The average plaque counts are computed from the two runs. The % plaque reduction is calculated, for example, as (Naïve_avg-Imm3_avg)/Naïve_avg. The sera are also tested for ELISA reactivity to recombinant MPV F protein. The endpoint titers will be determined for each animal. The % plaque reduction numbers is expected to show a modest linear correlation with the ELISA titers.

These data are expected to demonstrate that macaque immunization with the MPV epitope-scaffold monomers or the MPV epitope-scaffold presented on HepBcAg particles may result in the production of MPV F-binding antibodies and MPV neutralizing antibodies. The % neutralization (% plaque reduction) is expected to be as high as 88% for HepBcAg particle-displayed MPV epitope-scaffolds, and as high as 72% for monomeric MPV epitope-scaffold. The average % plaque reduction for HepBcAg particle-presented scaffolds is expected to be higher than the average for any of the monomer samples.

All publications, patents and patent applications referenced in this specification are indicative of the level of ordinary skill in the art to which this application pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

1. An isolated polypeptide comprising an amino acid sequence according to (a) SEQ ID NO: 1; (b) one of SEQ ID NO: 17, SEQ ID NO: 18 and SEQ ID NO:19; or (c) one of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, and SEQ ID NO: 42.

2. The isolated polypeptide of claim 1, wherein the polypeptide includes a sequence selected from the group consisting of SEQ ID NOS: 2-4.

3. The isolated polypeptide of claim 1, wherein the polypeptide includes a sequence having at least 90% amino acid sequence identity to a sequence selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 23, SEQ ID NO: 24, SEQ ID NO: 25, SEQ ID NO: 26, SEQ ID NO: 27, and SEQ ID NO: 28.

4. The isolated polypeptide of claim 1, wherein the polypeptide includes a sequence selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 23, SEQ ID NO: 24, SEQ ID NO: 25, SEQ ID NO: 26, SEQ ID NO: 27, and SEQ ID NO: 28.

5. The isolated polypeptide of claim 1, comprising an amino acid sequence according to at least one of SEQ ID NO: 17, SEQ ID NO: 18 and SEQ ID NO:19.

6. The isolated polypeptide of claim 5, wherein the polypeptide includes a sequence having at least 90% amino acid sequence identity to a sequence selected from the group consisting of SEQ ID NO: 20, SEQ ID NO: 21 and SEQ ID NO: 22.

7. The isolated polypeptide of claim 5, wherein the polypeptide includes a sequence selected from the group consisting of SEQ ID NO: 20, SEQ ID NO: 21, and SEQ ID NO: 22.

8. An isolated polypeptide comprising an amino acid sequence having at least 90% amino acid sequence identity to a sequence selected from the group consisting of SEQ ID NO: 29, SEQ ID NO: 30, SEQ ID NO: 31, SEQ ID NO: 32, SEQ ID NO: 33, SEQ ID NO: 34, SEQ ID NO: 35, and SEQ ID NO: 36.

9. An isolated polypeptide comprising an amino acid sequence according to at least one of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, and SEQ ID NO: 42.

10. A virus-like particle (VLP) comprising one or more of the polypeptide of claim 1.

11. An isolated nucleic acid encoding the polypeptide of claim 1.

12. A recombinant expression vector comprising the isolated nucleic acid of claim 11 operatively linked to a promoter.

13. A recombinant host cell comprising the recombinant expression vector of claim 12.

14. A pharmaceutical composition comprising the polypeptide of claim 1 and a pharmaceutically acceptable carrier.

15. A method for treating a metapneumovirus (MPV) infection, the method comprising administering to a subject infected with MPV an amount effective to treat the infection of the pharmaceutical composition of claim 14.

16. A method for limiting development of an MPV infection, the method comprising administering to a subject at risk of MPV infection an amount effective to limit development of an MPV infection of the pharmaceutical composition of claim 14.

17. A method for generating an immune response in a subject, the method comprising administering to the subject an amount effective to generate an immune response of the pharmaceutical composition of claim 14.

18. (canceled)

19. A method for monitoring an MPV-induced disease in a subject and/or monitoring response of the subject to immunization by an MPV vaccine, the method comprising contacting the polypeptide of claim 1, with a bodily fluid from the subject and detecting MPV-binding antibodies in the bodily fluid of the subject.

20. The method of claim 19, wherein the bodily fluid includes serum or whole blood.

21-24. (canceled)

25. A method of preventing an MPV infection, the method comprising administering to a subject at risk of MPV infection an amount effective to prevent the development of an MPV infection of the pharmaceutical composition of claim 14.
